(19) United States
(12) Patent Application Publication
(10) Pub. No.: US 2002/0015723 A1
(43) Pub. Date: Feb. 7, 2002

(54) Diabetic diet management system

(76) Inventor: Donna L. Koenig, Edwardsville, IL (US)

Correspondence Address: POLSTER, LIEDER, WOODRUFF & LUCCHESI, 763 SOUTH NEW BALLAS ROAD, ST. LOUIS, MO, 63141-8750, US

(21) Appl. No.: 09/833,822

(22) Filed: Apr. 12, 2001

Related U.S. Application Data
(60) Provisional application No. 60/196,729, filed on Apr. 13, 2000.

(51) Int. Cl. G06F 17/60; A61K 47/00

(52) U.S. Cl. 424/439; 705/2

(57) ABSTRACT

A diet management system and apparatus is described by which a patient on a prescribed diet can keep track of the portions or servings of various food groups consumed over the course of a day. The diet management apparatus includes a series of color-coded Exchange Cards or other exchange tokens for each permitted portion of a given food group on a prescribed diet with which the patient keeps track of the consumption of exchanges throughout the day. A booklet is provided for organizing, storing and using the Exchange Cards according to the system.

### CROSS-REFERENCE TO RELATED APPLICATIONS

This application claims priority to U.S. provisional patent application No. 60/196,729, filed Apr. 13, 2000.

### BACKGROUND OF THE INVENTION

The invention relates generally to the management of a specialized diet and, more specifically, to a method and apparatus for the daily management of a prescribed diet, such as a diabetic diet.

It has long been known that one major aspect of the successful control of diabetes mellitus, either Type I or Type II, is the long term control and management of the patient's diet. Generally speaking, a patient diagnosed with either type of diabetes mellitus is placed on a prescribed or specified diet based upon the patient's weight (or need to lose or gain weight), nutritional needs, average blood sugar levels and target blood sugar levels. Often times the treating physician refers the patient to a registered dietician for diet design and counseling with these parameters in mind.

Traditionally health care professionals believed that reduction of the patient's daily consumption of sugar was the mainstay of a diabetic diet. However, in recent years, health care professionals, including physicians and dieticians, have recognized that the patients should continue on a balanced diet, that is, a diet including meats, vegetables, dairy products, fats and starches (carbohydrate) Within the balanced diet, the patient is directed to consumer predetermined servings or “exchanges” from each of these food groups in a day, with a prescribed limit on servings of carbohydrates, which are implicated in high blood sugar. Generally speaking, one serving or exchange of a carbohydrate includes one serving containing approximately 15 grams of carbohydrate. By way of example, one-half of a plain bagel or one slice of bread would constitute on serving or exchange of carbohydrate.

The failure or inability of a patient to comply with the demands of his or her diet is a reason some patients do not achieve maximum control of their disease. Often it is difficult for the patient to keep track of the servings of a particular food group consumed over the course of a day. Or, on the other hand, the patient may consume of his or her allotted servings of a particular food group, for example meat, early in the day, requiring the patient to eat a less desirable or less palatable meal or snack late in the day.

Therefore, it would be advantageous to have a method and apparatus by which a diabetic easily keep track of his or her consumed food portions during the course of a day and thereby remain on a prescribed diet.

### SUMMARY OF THE INVENTION

It is among the objects of the invention to provide a diet management system and apparatus by which a patient on a prescribed diet can keep track of the portions or servings of various food groups consumed over the course of a day.

Another object of the present invention is to provide such a diet management system which include a series of Exchange Cards or other exchange tokens for each permitted portion of a given food group on a prescribed diet with which the patient can keep track of the consumption of exchanges throughout the day.

Still another object of the invention provides for tabulation of the Exchange Cards or tokens for each permitted portion of a given food group on a prescribed diet which allows the patient to determine, at any time during the day, the amount of food already consumed in a given food group and determine the amount of food the patient may yet consume that day in any given food group, based upon a prescribed diet.

More specifically, in one aspect, one embodiment the present invention includes a method for managing a diet for an individual, including providing multiple exchange tokens, which in one embodiment are Exchange Cards, wherein each Card or token represents a dietary exchange according to the diet, providing a first location for placing a total daily allotment of exchange tokens according to the diet, and providing a second location to which the exchange tokens are transferred during the day as the individual consumes food, to keep track of food exchanges consumed during the day.

In an exemplary embodiment, the method further includes providing a listing of the daily allotment of food exchanges according to the diet, instructing the individual to select a group of exchange tokens from the plurality of exchange tokens, wherein the group of exchange tokens corresponds to the daily allotment of food exchanges, and instructing the individual to place the group of exchange tokens representing the daily allotment in the first location, and further instructing the individual to transfer exchange tokens representing each food consumed from the first location to the second location as the food is consumed during the day. The method can further include instructing the user to transfer individual exchange tokens from the first location to the second location as food is consumed during a day comprises providing the user with a list of foods and their corresponding exchanges according to the diet. Still further, the method can include providing the plurality of exchange tokens as a plurality of different groups of exchange tokens, each of the groups representing a different type of food exchange according to the diet. In a preferred embodiment, for example, the method includes providing a plurality of different groups of Exchange Cards: a group of FRUIT Exchange Cards, a group of MILK Exchange Cards, a group of STARCH Exchange Cards, a group of VEGETABLE Exchange Cards, a group of FAT Exchange Cards, and a group of MEAT & SUBSTITUTES Exchange Cards.

In another aspect, the present invention includes apparatus for managing a diet, the apparatus comprising a first plurality of exchange tokens, each token marked or otherwise configured to represent a dietary exchange according to the diet, a first location configured to receive a second plurality of exchange tokens representing a total daily allotment of dietary exchanges according to the diet, and a second location configured to receive the second plurality of exchange tokens. In an exemplary embodiment of the apparatus, the exchange tokens are the Exchange Cards, and the first and second locations are, respectively, a first pocket and a second pocket coupled by a binder to form a booklet, and each configured to receive the Exchange Cards. The cards are color-coded according to the food group.

Thus, in accordance with the invention, an apparatus and method of using the apparatus (together referred to as a management system), for managing a prescribed diet is provided, as well as a method of managing a diet for an individual by providing the apparatus and instructing the individual is described.

By way of example, if a patient is placed on a prescribed 1500 kcal per day diet, she typically would be allowed six (6) starches, five (5) meats, three (3) vegetables, three (3) fruits, three (3) milks, and five (5) fats. Therefore, at the beginning of the day, 6 starch cards, 5 meat cards, 3 vegetable cards, 3 milk cards and 5 fat cards are placed in the first pocket of the holder. When the patient consumed a portion she moves a corresponding card to the second or “used” pocket. For example, if she has one cup of milk, one scrambled egg and a piece of toast for breakfast, she would removed one milk card, one meat card and one starch card from the first pocket and place in the second pocket. The patient repeats this routine for subsequent snacks and meals. Hence, at any time during a day patient can calculate the amount of each food consumed and the amount the patient can consume over the remainder of the day.

The apparatus can include directions for use, illustrated example diets, and an index of foods and food groups. Moreover, each individual exchange card can include a listing of the most often consumed foods within that group as well as portion size. For example, a STARCH card can contain a listing such as “Animal Cracker-8”; “Bagel-½ or 1 oz.”; etc. to aid the patient in food choices.

### BRIEF DESCRIPTION OF THE DRAWINGS

FIG. 1 is a front view of a booklet open to a front page in accordance with one embodiment of the present invention;

FIG. 2 is a front view of the booklet open to a back cover;

FIG. 3 is a front surface view of an Exchange Card from a first group of cards in accordance with one embodiment of the present invention;

FIG. 4 is a rear surface view of the Exchange Card shown in FIG. 3;

FIG. 5 is a front surface view of an Exchange Card from a second group of cards in accordance with one embodiment of the present invention;

FIG. 6 is a front surface view of an Exchange Card from a third group of cards in accordance with one embodiment of the present invention;

FIG. 7 a rear surface view of the Exchange Card shown in FIG. 6;

FIG. 8 is a front surface view of an Exchange Card from a fourth group of cards in accordance with one embodiment of the present invention;

FIG. 9 is a rear surface view of the Exchange Card shown in FIG. 8;

FIG. 10 is a front surface view of an Exchange Card from a fifth group of cards in accordance with one embodiment of the present invention;

FIG. 11 is a rear surface view of the Exchange Card shown in FIG. 10;

FIG. 12 is a front surface view of an Exchange Card from a sixth group of cards in accordance with one embodiment of the present invention;

FIG. 13 is a rear surface view of the Exchange Card shown in FIG. 12;

FIG. 14 is a front surface view of a first auxiliary card in accordance with one embodiment of the present invention;

FIG. 15 is a rear surface view of the auxiliary card shown in FIG. 14;

FIG. 16 is a front surface view of a second auxiliary card in accordance with one embodiment of the present invention;

FIG. 17 is a front surface view of a third auxiliary card in accordance with one embodiment of the present invention;

FIG. 18 is a front surface view of a fourth auxiliary card in accordance with one embodiment of the present invention;

FIG. 19 is a front surface view of a fifth auxiliary card in accordance with one embodiment of the present invention; and

FIG. 20 is a flow chart illustrating the steps of a method in accordance with one embodiment of the present invention.

### DESCRIPTION OF THE PREFERRED EMBODIMENT

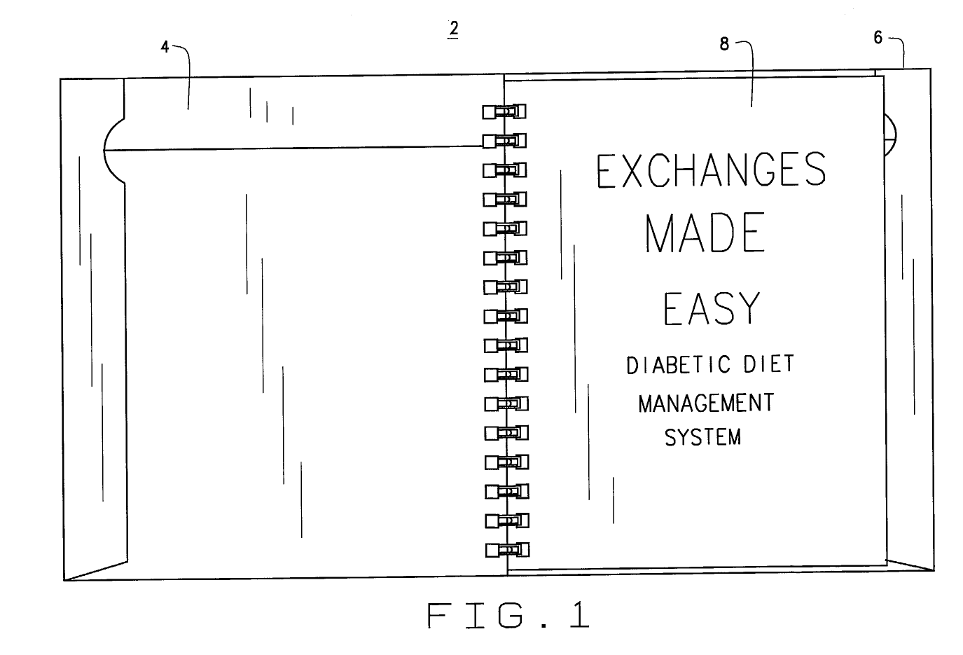

The apparatus and method of use thereof for the management of a specialized diet is best understood by first describing the apparatus. Referring to the figures, FIG. 1 shows a first front view of an open booklet 2, having a first or front outer cover 4 and a second or back outer cover 6, with a plurality of pages 8 in between. In one embodiment, when closed, the booklet 2 is of a size suitable for fitting into a shirt or jacket pocket for convenient portability. Alternatively, the booklet 2 can be larger. In an exemplary embodiment, the front outer cover 4 is constructed with an inner pocket flap 10 folded and secured against the front outer cover 4. Thus, flap 10 forms a first or front pocket 12 that is accessible from the inner surface of the front outer cover 4.

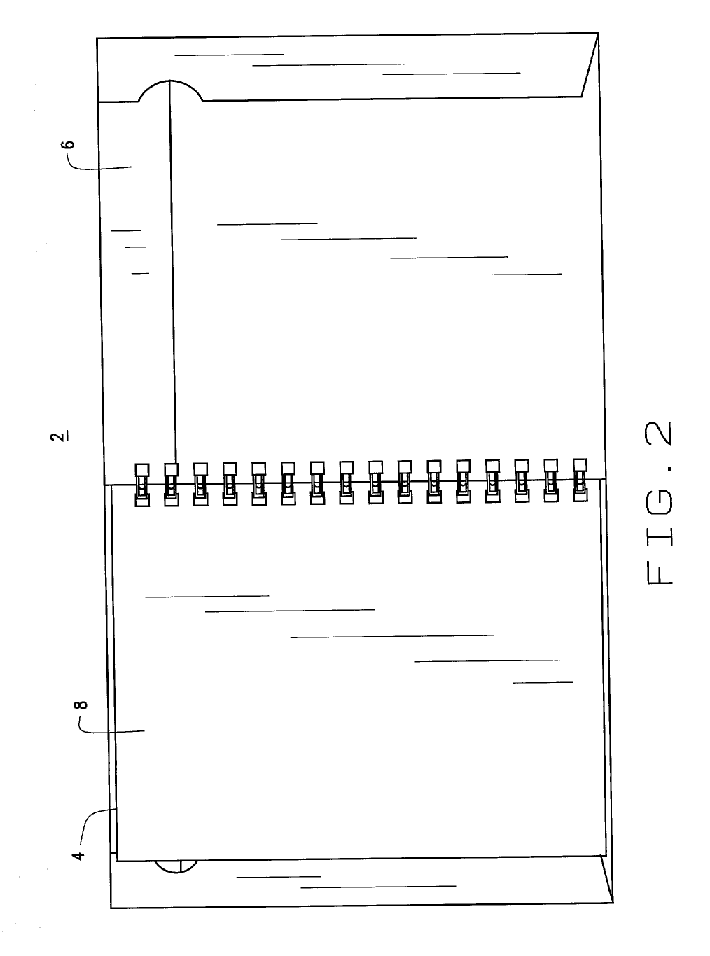

FIG. 2 shows a second front view of the booklet 2, open to the second or back or outer cover 6. The back outer cover 6 is also constructed with a pocket flap 14 folded and secured against the back outer cover 6. Thus, flap 14 forms a second or back pocket 16 that is accessible from the inner surface of the back outer cover 4. In an exemplary embodiment as shown in FIGS. 1 and 2, the first and second pockets are coupled to a binder such as a spiral to form the booklet 2, but alternatively the pockets can be formed from a single sheet of material and may take the form of a folder or pamphlet type cover. For durability, the booklet 2 may be fashioned form a suitable plastic material, or laminated.

The plurality of pages 8 can contain any number of pages, and the pages can display a variety of educational material or data and may be organized in groups or sections in different combinations. For example, in an exemplary embodiment, the pages 8 include a first group of several pages bearing background information on diabetic diets, a second group of pages with directions for use of the apparatus in the novel management system, and a third group of pages displaying an index that correlates foods to their corresponding food groups. A fourth group of pages lists foods that are classified as combination foods as described below. The different groups of pages can be color-coded or otherwise coded with dividers, tags or markers to make finding specific information more convenient. In an exemplary embodiment, the pages include pages with the directions for use but otherwise, any other written material can be included or substituted in the booklet 2 and remain within the scope of the invention.

The apparatus also includes a plurality of cards that will be referred to as Exchange Cards. The Exchange Cards are so called because each card represents a single dietary “exchange” in a dietary or nutritional plan. In an exemplary embodiment, the foods and groups are organized into exchanges according to a dietary or nutritional plan for a diabetic person. However, the organization of foods into groups and exchanges can be varied to accommodate different dietary or nutritional plans.

In one embodiment, the cards include multiple groups or stacks of cards, each group corresponding to a specific food group, with one group or stack of cards for each food group. The food groups are, for example, VEGETABLE; MEAT & SUBSTITUTES; FAT; STARCH; FRUIT; and MILK. However, in alternative embodiments, any system of dietary classification on which a dietary or nutrition plan is based can be used, and the precise division and naming of the food groups can vary accordingly. For example, different health or medical conditions may require different dietary restrictions or nutritional plans that vary in terms of restricted or unrestricted foods. Accordingly, the present invention contemplates adapting the system described herein by, for example, varying the number and types of food groups and thus card groups, and the foods and serving sizes listed on the Exchange Cards.

In alternative embodiments, exchange tokens other than cards may be used to represent a serving of food from each food group. For example, magnetic tokens, stickers, electronic tokens in an electronic system such as a hand-held computer or the like may be used. Any such token need only be marked or otherwise configured in some way as to identify the type of food exchange, according to the diet, that the token represents.

In an exemplary embodiment, the Exchange Cards are used and each card in a group is identical. For example, the Exchange Cards in one group each bear a list of commonly available foods within the given food group, and for each food is listed a serving size. In one embodiment, the different groups of cards are color-coded or otherwise coded, labeled or tagged for easy identification when cards from different food groups are stacked together.

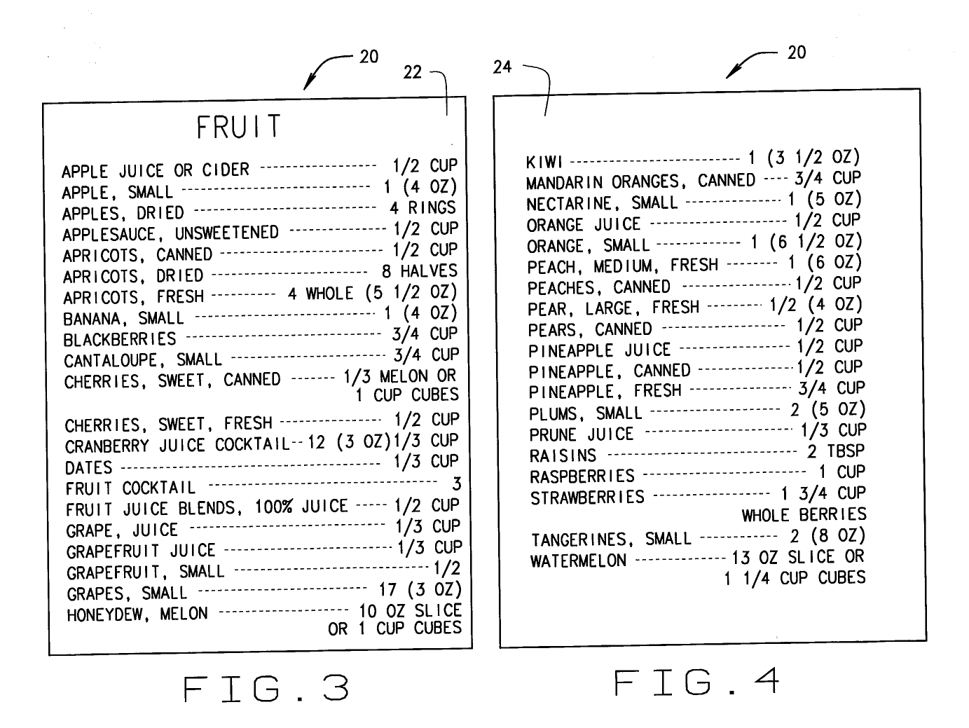

For example, referring again to the figures, FIG. 3 is a front surface view of an Exchange Card 20 from a first group of the Exchange Cards. Card 20 is a FRUIT Exchange Card, on the front surface 22 of which is printed the first portion of a list of commonly available fruits, and a corresponding serving size for each fruit listed. FIG. 4 is a rear surface view of the same card 20, showing the rear surface 24 of card 20 on which is printed a second and final portion of the list started on the front surface 22.

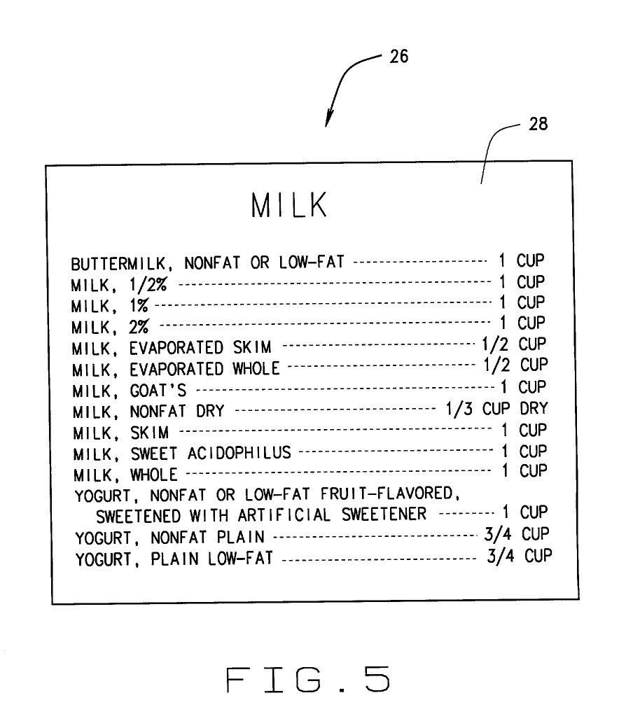

FIG. 5 is a front surface view of an Exchange Card 26 from a second group of the Exchange Cards. Card 26 is a MILK Exchange Card, on the front surface 28 of which is printed a list of commonly available milk or dairy foods, and a corresponding serving size for each milk or dairy food listed.

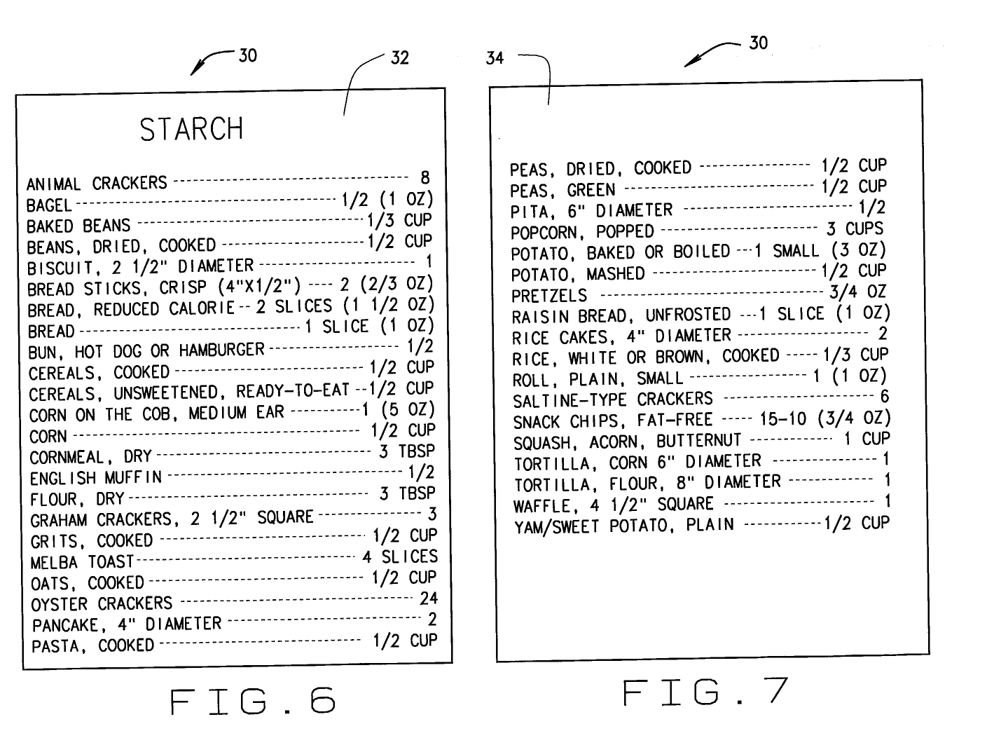

FIG. 6 is a front surface view of an Exchange Card 30 from a third group of the Exchange Cards. Card 30 is a STARCH Exchange Card, on the front surface 32 of which is printed the first portion of a list of commonly available foods that are typically classified as starches in dietary or nutritional plans, and a corresponding serving size for each food listed. FIG. 7 is a rear surface view of the same card 30, showing the rear surface 34 of card 30 on which is printed a second and final portion of the list started on the front surface 32.

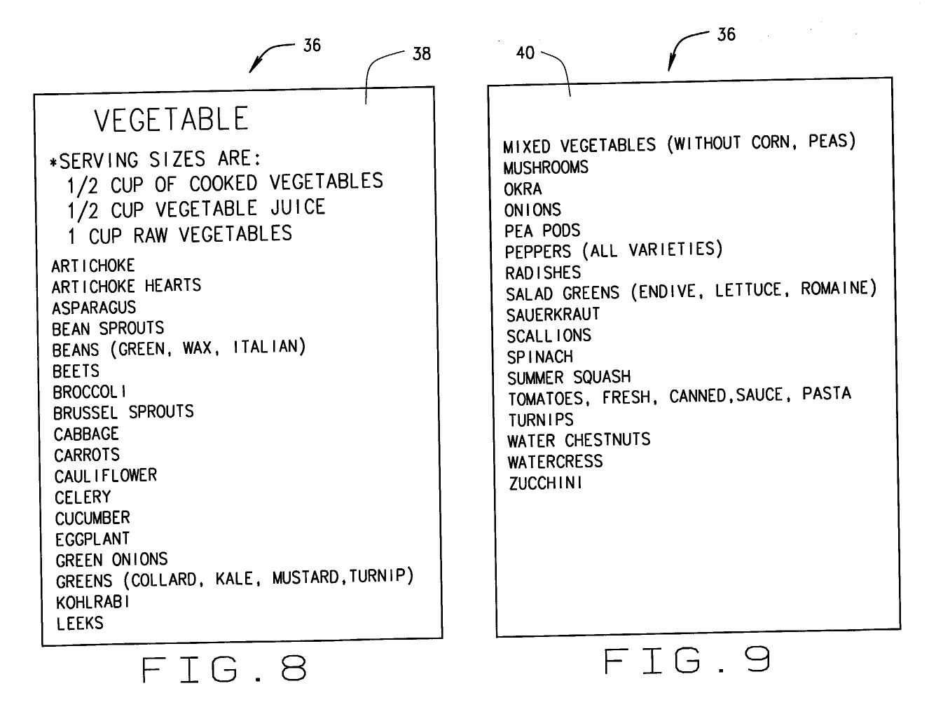

FIG. 8 is a front surface view of an Exchange Card 36 from a fourth group of the Exchange Cards. Card 36 is a VEGETABLE Exchange Card, on the front of which is printed the first portion of a list of vegetables, and a corresponding serving size for each vegetable listed. FIG. 9 is a rear surface view of the same card 36, showing the rear surface 40 of card 36 on which is printed a second and final portion of the list started on the front surface 38.

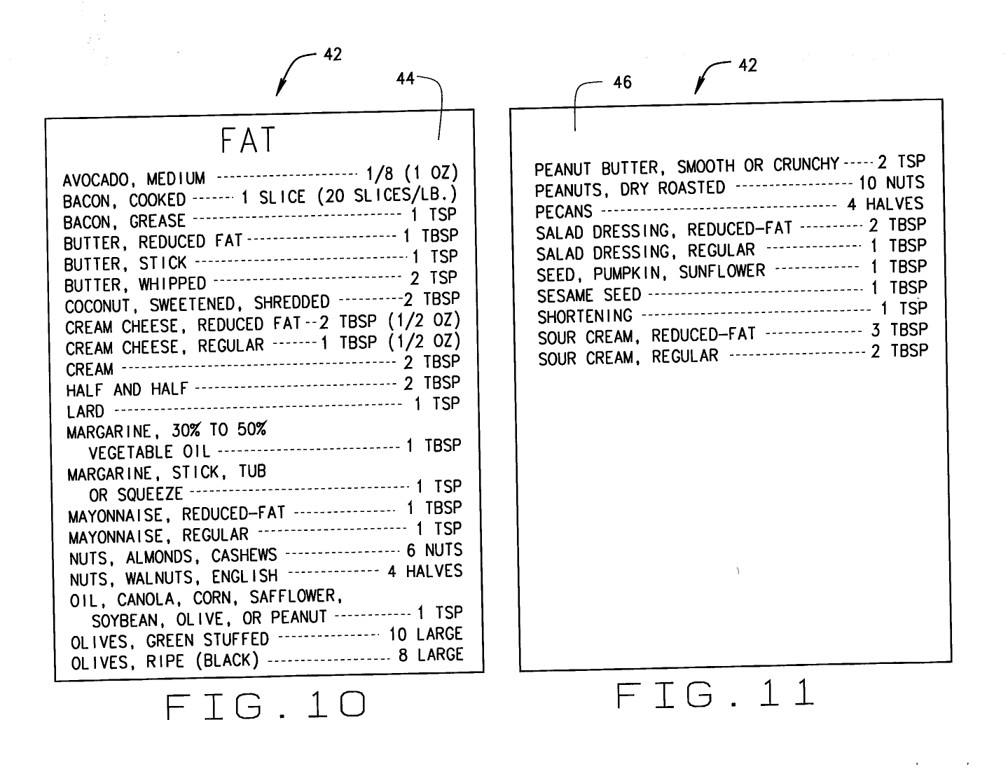

FIG. 10 is a front surface view of an Exchange Card 42 from a fifth group of the Exchange Cards. Card 42 is a FAT Exchange Card, on the front surface 44 of which is printed the first portion of a list of commonly available foods that are typically classified as fats in dietary or nutritional plans, and a corresponding serving size for each food listed. FIG. 11 is a rear surface view of the same card 42, showing the rear surface 46 of card 42 on which is printed a second and final portion of the list started on the front surface 44.

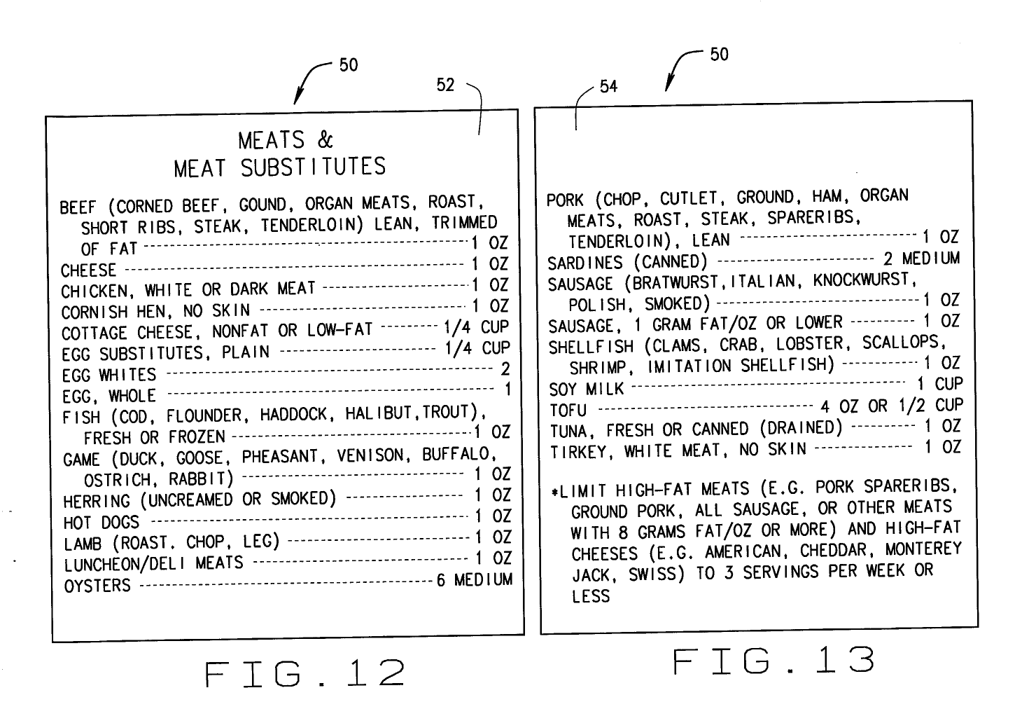

FIG. 12 is a front surface view of an Exchange Card 50 from a sixth group of the Exchange Cards. Card 50 is a MEAT & SUBSTITUTES Exchange Card, on the front surface 52 of which is printed the first portion of a list of commonly available foods that are typically classified as meats or meat substitutes in a dietary or nutritional plan, and a corresponding serving size for each food listed. FIG. 13 is a rear surface view of the same card 50, showing the rear surface 54 of card 50 on which is printed a second and final portion of the list started on the front surface 52.

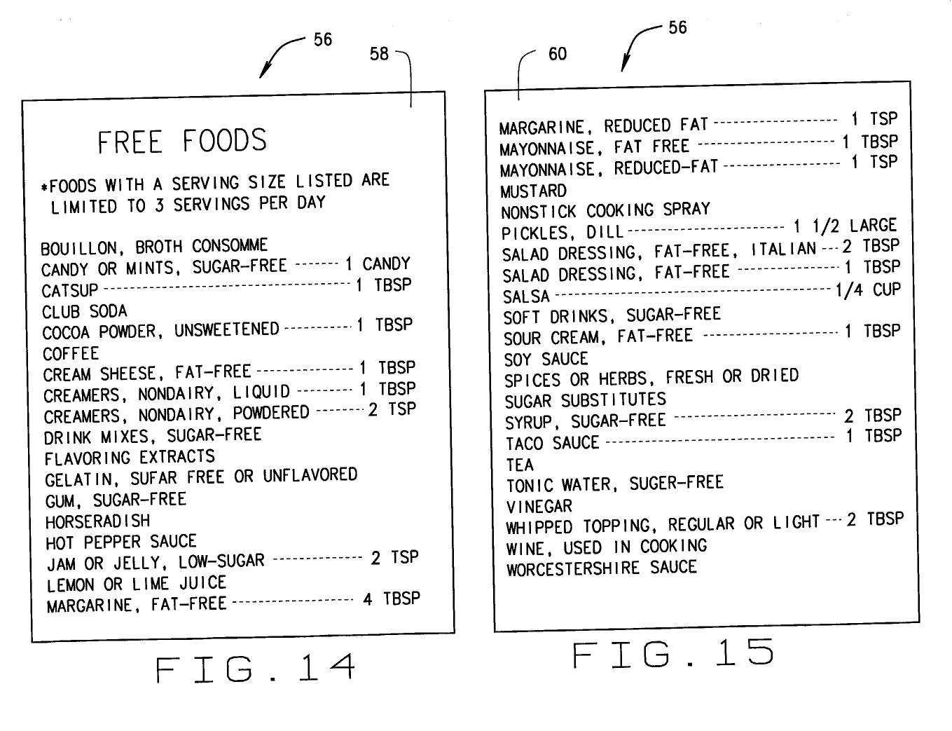

FIG. 14 is a front surface view of a FREE FOODS auxiliary card 56 on the front surface 58 of which is printed the first portion of a list of commonly available foods that are typically classified as “free foods” in dietary or nutritional plans because of their very low caloric and sugar contents, and a corresponding serving size for each such food listed. FIG. 15 is a rear surface view of the same card 56, showing the rear surface 60 of card 56 on which is printed a second and final portion of the list started on the front surface 58. In an exemplary embodiment, only one FREE FOODS auxiliary card 56 is included and used because such foods frequently do not require the careful monitoring of consumption that foods from the other food groups do require. However, in alternative embodiments, when consumption of free foods must be restricted because of, for example, the salt content of certain such foods, or where for any other reason the intake of such free foods is to be monitored, multiple FREE FOODS cards 56 are provided.

As should be clear, for the system to work in accordance with the present invention, each and every food corresponding to a food in a food group generally appears only once, and in one food group. However, in a given dietary or nutritional plan, some foods will be combination foods. A combination food is one that may be counted alternatively as one of several different food groups, or as a combination of multiple food groups. In some dietary plans, such as the exemplary diabetic dietary plan, processed foods such as cakes and cookies, ice cream, and snack chips are examples of combination foods. For example, in an exemplary diabetic nutritional plan, a serving of sherbet can be counted alternatively as a STARCH, a FRUIT or a MILK exchange. A serving of tortilla chips is counted as two exchanges of FAT, plus, alternatively, one exchange of a STARCH, a FRUIT or a MILK.

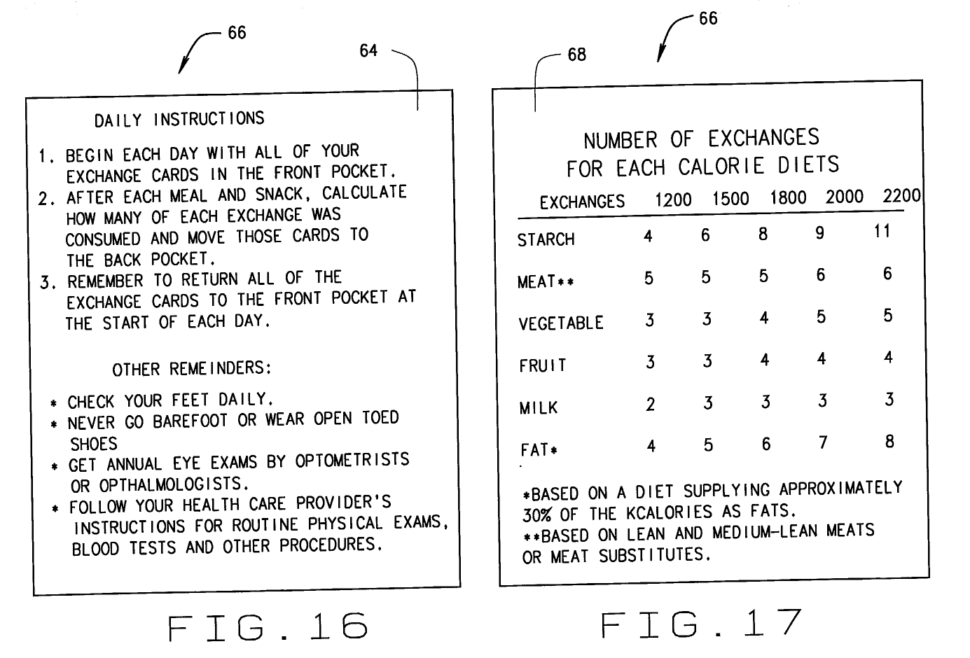

FIG. 16 is a front surface view of a second auxiliary card 62, a Daily Instructions card. On the front surface 64 of card 62 is printed, for example, a summary of steps for using the system, and other such reminders or helpful hints.

FIG. 17 is a front surface view of a third auxiliary card 66, a Number of Exchanges For Various Calorie Diets card. On the front surface 68 of card 66 is printed, for example, a table displaying, the number of allotted exchanges from each food group that will amount approximately to a specified number of calories consumed for the day. For example, as shown in FIG. 17, given a dietary plan for managing diabetes the number of allotted exchanges from each food group for a 1200 calorie/day diet, a 1500 calorie/day diet, an 1800 calorie/day diet, a 2000 calorie/day diet and a 2200 calorie/day diet are listed. It should be appreciated that the exchange allotments for each food group, as listed in such a table, will vary depending on the dietary or nutritional plan to which the system and method are being applied.

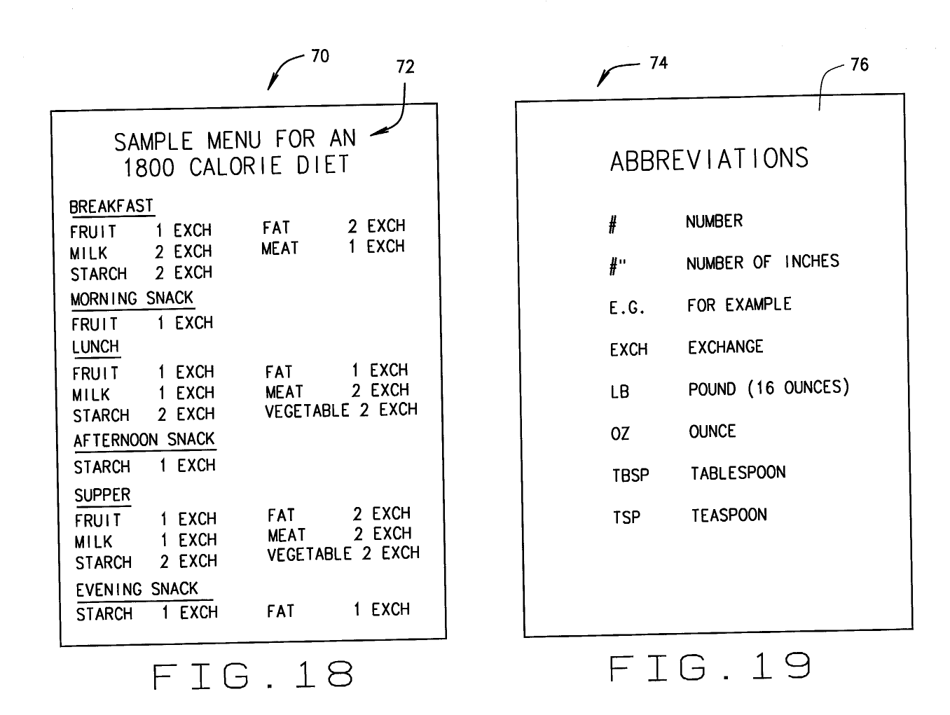

FIG. 18 is a front surface view of a fourth auxiliary card 70, a Sample Menu card. On the front surface 72 of card 70 is printed, for example, a sample menu for an 1800 calorie/day diet, with allotted dietary exchanges from each food group listed for each meal during the day. The printed menu on card 70 is adapted for individuals following a higher- or lower- calorie diet. In one embodiment, for example, multiple such cards 70 are provided, each with a different preprinted menu for each diet that differs in total calorie content and thus allotted exchanges. The card 70 that corresponds to the planned daily caloric consumption is then used. It will again be appreciated that all such sample menus and corresponding exchanges will vary according to the specific dietary or nutritional plan to which the system and method are being applied.

FIG. 19 is a front surface view of a fifth auxiliary card 74 on the front surface 76 of which is printed a list of abbreviations used on the other cards. Listed are abbreviations that are used to indicate the weight or volume of a food that corresponds to one exchange in the dietary or nutritional plan, and any other abbreviations for which such a reference might be helpful. It will be appreciated by those skilled in the art that the information contained on the cards illustrating the Number of Exchanges For Various Calorie Diets, Abbreviations, and a Sample Menu can be provided, in alternative embodiments, on pages within the booklet 2 and not on cards. The only cards essential to the exemplary embodiment are the food Exchange Cards.

### Method of Use

Referring again to FIG. 18, and card 70 with the Sample Menu For an 1800 Calorie Diet, it will be appreciated that a patient's caloric intake is spread over the course of a day, including breakfast, a morning snack, lunch, afternoon snack, supper and evening snack.

As shown in FIG. 18 and the Sample Menu For an 1800 Calorie Diet, it will be observed that over the course of the day, for a representative 1800 calorie diet, the patient is allotted 6 starches, 5 meats, 3 vegetables, 3 fruits, 3 milks, and 5 fats. Hence, the patient will require the following Exchange Cards: 6 STARCH, 5 MEAT & SUBSTITUTES, 3 VEGETABLE, 3 FRUIT, 3 MILK, and 5 FAT.

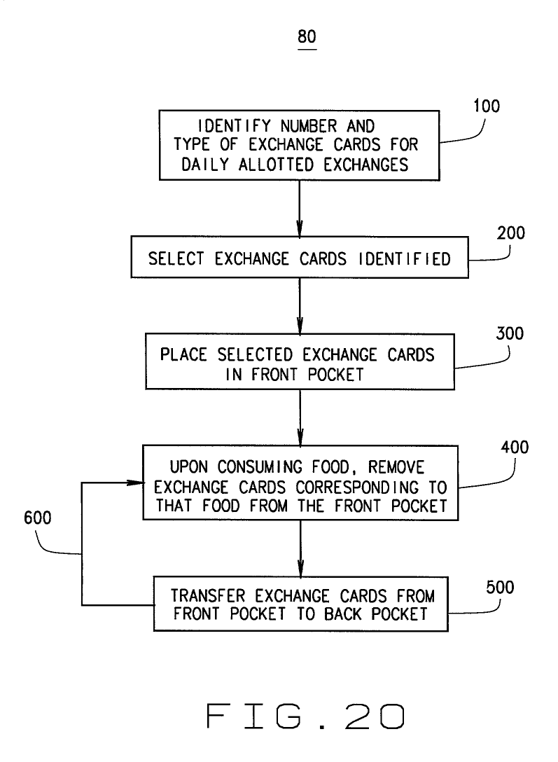

FIG. 20 is a flow chart of a dietary management method 80 in accordance with an exemplary embodiment of the invention. Thus, to practice the method 80, at the beginning of the day the patient identifies 100 the number and type of each Exchange Card for each of the allotted exchanges for each food group according to the selected diet, for example the 1800 Calorie Diet shown on card 70. The identification of exchanges thus entails the step of identifying or selecting a particular diet or dietary plan. In some cases, the selected diet or dietary plan may vary from day to day, and thus the daily allotment of exchanges will vary accordingly. The patient then selects 200 the total number and type of Exchange Cards so identified, and places or positions 300 all the Exchange Cards in the first or front pocket in the front cover 4 of the booklet 2. Upon consuming a food from a designated food group, or a combination food as described above, the patient removes 400 from the front pocket an Exchange Card or combination of cards corresponding to the exchange or exchanges for that food, and transfers 500 the Exchange Card or cards from the front pocket in the front cover 4 to the second or back pocket in the back cover 6. For example, if the patient has a breakfast consisting of a small banana, a cup of milk, a bagel, and a scrambled egg, she would transfer one FRUIT, one MILK, two STARCH and one MEAT & SUBSTITUTES Exchange Cards from the front pocket to the rear pocket.

Steps 400 and 500 are repeated 600 throughout the day, and the stack of Exchange Cards in the first pocket dwindles as the stack in the second pocket grows. It will be appreciated that at any time during the day, at supper for example, the patient can consult the booklet and by noting the number and type of cards remaining in the front pocket determine what foods from which groups, and how many exchanges, she can have for supper. It will be appreciated that the apparatus described above can be pre-printed or manufactured and provided to individual patients or other individuals with instructions on how to use the apparatus according to method 80. The instructions may be verbal from a health or nutrition professional or may be included on an auxiliary card such as card 62, or on a page within booklet 2.

The foregoing system and apparatus eliminates the problem of forgetting what was consumed earlier in the day and trying to determine what the patient can eat later in the day. Furthermore, it is much more convenient and less time consuming than documenting each food eaten in a food diary or the like, thereby encouraging patient compliance.

Although the diet management system of the present is illustrated in reference to diabetic diets, those skilled in the art will appreciate that it can be adapted for use with other specialized diets such as high protein, low-fat, low cholesterol or others, without departing from the scope of the invention. Moreover, although the apparatus described above is the best and most cost effective mode of working the invention now known to the inventor, it may be possible to incorporate the system into a magnetic form using magnetic tokens for the Exchange Cards, or an electronic form, such as a computer program for use with a conventional or a hand-held computer, which is programmed with the electronic equivalents of the illustrated Exchange Cards which can be moved from an “available” file to a “consumed” file electronically. Such a system could be programmed to provide an immediate read-out of exchanges consumed and exchanges available to the patient.

Since it will be appreciated that various changes and modifications can be made in the invention without departing from the scope of the invention, the foregoing description and the accompanying drawings are intended to be illustrative only and should not be viewed in a limiting sense.

What is claimed is:

1. A method for managing a diet for an individual, said method comprising:
providing a plurality of exchange tokens, each token representing a dietary exchange according to the diet;
providing a first location for placing a total daily allotment of exchange tokens according to the diet; and
providing a second location to which the exchange tokens are transferred during a day as food is consumed to keep track of food exchanges consumed during the day.

2. A method for managing a diet in accordance with claim 1, said method further comprising:
providing a listing of the daily allotment of food exchanges according to the diet;
instructing the individual to select a group of exchange tokens from the plurality of exchange tokens, wherein the group of exchange tokens corresponds to the daily allotment of food exchanges;
instructing the individual to place the group of exchange tokens representing the daily allotment in the first location; and
instructing the individual to transfer exchange tokens representing each food consumed from the first location to the second location as the food is consumed during the day.

3. A method for managing a diet in accordance with claim 2 wherein instructing the user to transfer individual exchange tokens from the first location to the second location as food is consumed during a day comprises providing the user with a list of foods and their corresponding exchanges according to the diet.

4. A method for managing a diet in accordance with claim 1 wherein providing a plurality of exchange tokens comprises providing a plurality of different groups of exchange tokens, each of the groups representing a different food exchange according to the diet.

5. A method for managing a diet in accordance with claim 1 wherein providing a plurality of exchange tokens comprises providing a plurality of Exchange Cards.

6. A method for managing a diet in accordance with claim 5 wherein providing a plurality of Exchange Cards comprises providing a plurality of different groups of Exchange Cards, each of the groups representing a different food exchange according to the diet.

7. A method for managing a diet in accordance with claim 6 wherein providing a plurality of different groups of Exchange Cards comprises providing a group of FRUIT Exchange Cards, a group of MILK Exchange Cards, a group of STARCH Exchange Cards, a group of VEGETABLE Exchange Cards, a group of FAT Exchange Cards, and a group of MEAT & SUBSTITUTES Exchange Cards.

8. A method for managing a diet in accordance with claim 7 wherein providing a first location for placing a total daily allotment of exchange tokens according to the diet and providing a second location to which the exchange tokens are transferred during a day as food is consumed to keep track of food exchanges consumed during the day, comprises providing for the first location a first pocket configured to receive the Exchange Cards and for the second location a second pocket configured to receive the Exchange Cards.

9. A method for managing a diet in accordance with claim 8 wherein providing the first pocket and the second pocket comprises providing the pockets coupled to a binder to form a booklet.

10. A method for managing a diet in accordance with claim 9 comprising providing the booklet in a size adapted to fit into a jacket pocket.

11. A method for managing a diet in accordance with claim 9 comprising providing the booklet in a size adapted to fit into a shirt pocket.

12. Apparatus for managing a diet, said apparatus comprising:
a first plurality of exchange tokens, each said token configured to represent a dietary exchange according to the diet,
a first location configured to receive a second plurality of exchange tokens representing a total daily allotment of dietary exchanges according to the diet; and
a second location configured to receive the second plurality of exchange tokens.

13. Apparatus in accordance with claim 12 wherein said first plurality of exchange tokens comprises a plurality of different groups of exchange tokens, each of said different groups representing a different food group according to the diet.

14. Apparatus in accordance with claim 12 wherein said first plurality of exchange tokens comprises a plurality of Exchange Cards.

15. Apparatus in accordance with claim 14 wherein said first plurality of Exchange Cards comprises a plurality of different groups of Exchange Cards, each of said different groups of Exchange Card groups representing a different food group according to the diet.

16. Apparatus in accordance with claim 15 wherein said Exchange Cards are color-coded according to the food group.

17. Apparatus in accordance with claim 16 wherein said first location is a first pocket configured to receive a plurality of Exchange Cards representing the total daily allotment of dietary exchanges according to the diet, and said second location is a second pocket configured to receive the plurality of Exchange Cards.

18. Apparatus in accordance with claim 17 wherein said first pocket and said second pocket coupled to a binder to form a booklet.

19. Apparatus in accordance with claim 18 wherein said booklet further comprises a plurality of pages coupled to the binder.

20. A method of using the apparatus of claim 16 comprising:
identifying a number and type of Exchange Cards representing a daily allotment of dietary exchanges according to a diet;
selecting the identified Exchange Cards;
placing the Exchange Cards in the first pocket;
upon consuming food, removing a number and type of Exchange Cards corresponding the food from the first pocket; and
transferring the Exchange Cards removed from the first pocket to the second pocket.
